(19) United States
(12) Patent Application Publication
(10) Pub. No.: US 2007/0143145 A1
(43) Pub. Date: Jun. 21, 2007

(54) Systems and methods for evaluating patient-specific information and providing patient management recommendations for healthcare providers

(76) Inventors: Roger DeLusignan, South San Francisco, CA (US); Jeffrey Davis, South San Francisco, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/655,721

(22) Filed: Jan. 19, 2007

Related U.S. Application Data
(63) Continuation of application No. 10/042,766, filed on Jan. 8, 2002, now abandoned.
(60) Provisional application No. 60/332,359, filed on Nov. 16, 2001.

(51) Int. Cl. G06Q 10/00; A61B 5/00

(52) U.S. Cl. 705/2; 600/300

(57) ABSTRACT

The present invention relates to the use of computerized systems for evaluating patient-specific data for the purpose of providing healthcare providers with recommendations for adhering to best medical practices.

### CROSS-REFERENCE TO RELATED APPLICATIONS

This application is a continuation of U.S. patent application Ser. No. 10/042,766, filed Jan. 8, 2002, which claims benefit of U.S. Provisional Patent Application No. 60/332,359, filed Nov. 16, 2001. The contents of each application listed in this paragraph are fully incorporated by reference herein for all purposes.

### TECHNICAL FIELD

This invention relates to the use of computerized systems for evaluating patient-specific data for the purpose of providing physicians with recommendations for adhering to best medical practices.

### BACKGROUND

Many medical associations and physician's groups publish recommendations for patient care that can be consulted by treating physicians and tailored for particular patients. However, such information is generally underutilized, because physicians do not have the time to evaluate recommendations on an individual basis for each patient under their care.

In order to facilitate managed patient care, computer systems have been implemented to provide patient-specific healthcare information to healthcare providers, such as physicians, nurses and others. In particular, may different automated systems have recently been implemented that identify individuals who are at risk for certain diseases and allow healthcare providers to intervene to manage patient care.

While these systems have proven to be useful for various purposes, they have primarily been designed as diagnostic, prognostic and treatment recommendation tools, rather than educational tools. Accordingly, tools are necessary for assessing a patient's status, from both the patient's and doctor's perspective, and then providing educational materials for the doctor which are customized for individual patients on an ongoing basis.

There are many publicly available government sponsored resources for evidence-based clinical practice guidelines. These resources are designed to facilitate consistent quality of care for patients with particular medical conditions by providing physician approved guidelines for patient care. For example, the National Guideline Clearinghouse provides a website for access to many such guidelines at www.guideline.gov. Other such guidelines are published by the Agency for Healthcare Research and Quality at www.ahrq.gov. In addition, many of the major professional associations publish their own guidelines, such as the American Heart Association and the American Diabetes Association.

These guidelines, also referred to herein as “recommendations” can be complex, depending on the particular medical condition for which they pertain. For example, the National Guideline Clearinghouse website provides a link to an article entitled “Prevention of Coronary Heart Disease in Clinical Practice” (Eur. Heart J. 10: 1434-1503 (1998). Among other information, these guidelines list six cardiovascular risk factors, seven primary intervention tools and four secondary intervention tools. Unfortunately, not all of these factors and tools relate to each patient, and the ability to quickly customize the guidelines for individual patients is necessary to make them useful in a clinical setting.

Accordingly, it is an object of the present invention to provide a computerized system for collecting and processing patient-specific information and generating customized recommendations for individual patients based on such input.

### DISCLOSURE OF THE INVENTION

The present invention relates to a method for providing patient-specific best medical practice recommendations for a population of patients suspected of having individual members with at least one preselected medical condition using a central system adapted to customize the recommendations for the individual members that comprises the steps of:
a) selecting a population of patients that is suspected of having a higher than normal risk for the preselected medical condition;
b) collecting personal patient information about the individual members that comprises factors that allow risk assessment of the preselected medical condition, and may also include information regarding factors that allow identification of the presence of the preselected medical condition;
c) processing the personal patient information by the central system to provide a patient analysis outcome that reflects risk assessment of the preselected medical condition;
d) collecting patient management information about the individual members that comprises a medical history relevant to the preselected medical condition; and
e) processing the patient management information by the central system to provide a recommendations outcome that reflects best medical practices for management and treatment of the individual members relative to the medical condition.

In a preferred embodiment, steps d) and e) are repeated at least once.

In one embodiment, the personal patient information and/or the patient management information can be obtained using an input collection tool, such as a form adapted for data collection.

The method can be adapted for use for any medical condition, including but not limited to coronary artery disease, diabetes, congestive heart failure, pulmonary disease, asthma, hypertension, depression, or any combination thereof.

Another aspect of the present invention is a system that is adopted for practicing the above-described method which comprises a computer with a central processing unit (CPU) that is programmed to receive and analyze patient-specific data to produce recommendations outcome.

### BRIEF DESCRIPTION OF THE DRAWINGS

FIG. 1 depicts a schematic representation of the system of the present invention.

FIG. 2 depicts a representative “Risk Assessment Survey” for Coronary Artery Disease that is used to collect personal patient information.

FIG. 3 depicts representative patient analysis outcome directed to a healthcare provider indicating the risk assessment of an individual fictitious patient.

FIG. 4 depicts a representative “Data Collection Tool” for Coronary Artery Disease that is used to collect patient management information.

FIG. 5 depicts a representative recommendations outcome directed to a healthcare provider indicating the generally accepted guidelines (i.e. recommendations) for ongoing treatment of an individual fictitious patient.

### MODES OF CARRYING OUT THE INVENTION

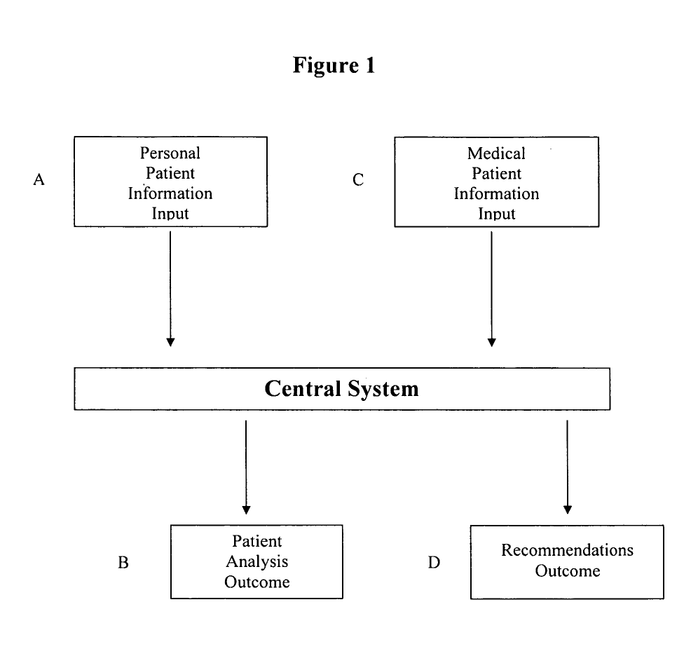

The present invention relates to a computerized system for collecting and processing patient-specific information and generating customized recommendations for individual patients based on such input. Computer systems can be represented generally as a systems for processing information from different sources to provide customized output. FIG. 1 is a simplified schematic representation of such a system according to the present invention which depicts the input aspect of the system on the top and the output aspect of the system on the bottom. The central system is programmed with information primarily from public sources regarding best practices for managing the care of patients with particular medical conditions (i.e., clinical practice guidelines), and the program is adapted to customize these best practices for individual patients.

The system requires a source of personal patient information and patient management information (collectively, “patient-specific information) which is input into the central system. After the information is input into the system, the information is processed according to the program specific for a particular medical condition. The initial output from the central system is an initial patient assessment called a “patient analysis outcome” based on the personal patient information. This outcome identifies the level of risk for the medical condition being contracted and may also identify the presence of the medical condition. The initial output is generally sent to the healthcare provider. As used herein, the term “healthcare provider” refers preferably to the patient's physician, most often their primary care physician, but may also refer to any employee, affiliate, colleague or agent of the physician or the healthcare provider's organization. For example, if the patient belongs to a health maintenance organization (HMO) the healthcare provider may refer to any employee of the HMO, such as doctors, specialists, nurses, administrators, pharmacy personnel, lab technicians, etc.

In addition, the system may require patient management information, which is usually obtained from the same healthcare provider that received the initial output. After this information is input into the central system, it is processed by the system, which then provides a recommendations outcome that relates to the best medical practices for management of the patient according to the clinical guidelines that are appropriate for that particular patient.

The methods of the present invention can be adapted for any preselected medical condition. Although coronary artery disease is the medical condition that is most thoroughly exemplified herein, it would be easy to adapt the system for other medical conditions, such as diabetes, congestive heart failure, pulmonary disease, etc.

Central System Hardware

The central system comprises a host computer adapted for receipt of patient-specific information either directly or indirectly via communication means. Such communication means include, inter alia, modem-mediated telecommunication, wireless telecommunication means, as well as information channeling from the source(s) of patient information to the central system via the internet.

The central system may be as simple a device as a personal computer, but preferably includes a file serve for storage of data from large patient populations. The file server may be directly connected to a personal computer (PC) including a screen, central processing unit (CPU), keyboard and printer, or may be accessible via the internet. In other words, the central system is not necessarily in one geographic location, but may actually consist of multiple pieces of hardware that are functionally associated via communication means. In one embodiment, the central system comprises at least one central server that is accessible on-line and at least one remote access terminal.

The processor of the central system is programmed with information relating to disease-specific clinical practice guidelines. Such information is generally obtained from publicly available sources as described elsewhere herein. The processor is also adapted for receiving and processing patient specific data from multiple sources. Computer-based data management systems are well known in the art and can easily be adapted for use as described herein.

The central system further comprises at least one output device that is adapted to provide both types of outcome discussed below. The output device may be a printer, fax machine, computer screen, etc., and may be adapted to send the output via mail, e-mail, internet, intranet, dedicated lines, etc. In addition, the output device may be programmed to generate outcome at preselected time periods. The output can take many different forms, including for example, components such as graphical element, textual elements, numerical elements and tabular elements.

Central System Software

The central system is pre-programmed prior to operation to analyze information specific to a preselected medical condition or conditions. Such programming includes the development of individual algorithms for processing patient-specific data to customize the output for the particular patient. For example, if the medical condition is coronary artery disease and the patient indicates they are not a smoker, then the algorithm may eliminate output information that concerns smoking.

Identifying a Population of Patients

The first step in the method of the present invention is selection of a patient population. Patient populations that are analyzed using the systems of the present invention are selected on the basis of criteria which is appropriate for the medical condition under study. This criteria is intended to narrow the general population in a way that is expected to include more members that are at risk, preferably at high risk, for the preselected medical condition. Accordingly, as used herein, the phrase “having a higher than normal risk for the preselected medical condition” means that the chosen population has a higher risk (e.g., >10%, preferably >20%) of having or developing the medical condition when compared to the general population. For example, since patients above the age of 45 are known to have a higher risk for coronary artery disease, the patient population being analyzed may include all patients over 45 years of age. In another example, if patients are being analyzed by the system because they are suspected of having diabetes, the patient population may consist of all patients having an age of 40 or above that have hypertension and/or are overweight. In yet another example, the selected patient population may be all patients of a specialist, such as a cardiologist, which by implication would be expected to have a higher risk for heart-related conditions such as coronary artery disease.

Personal Patient Information Input

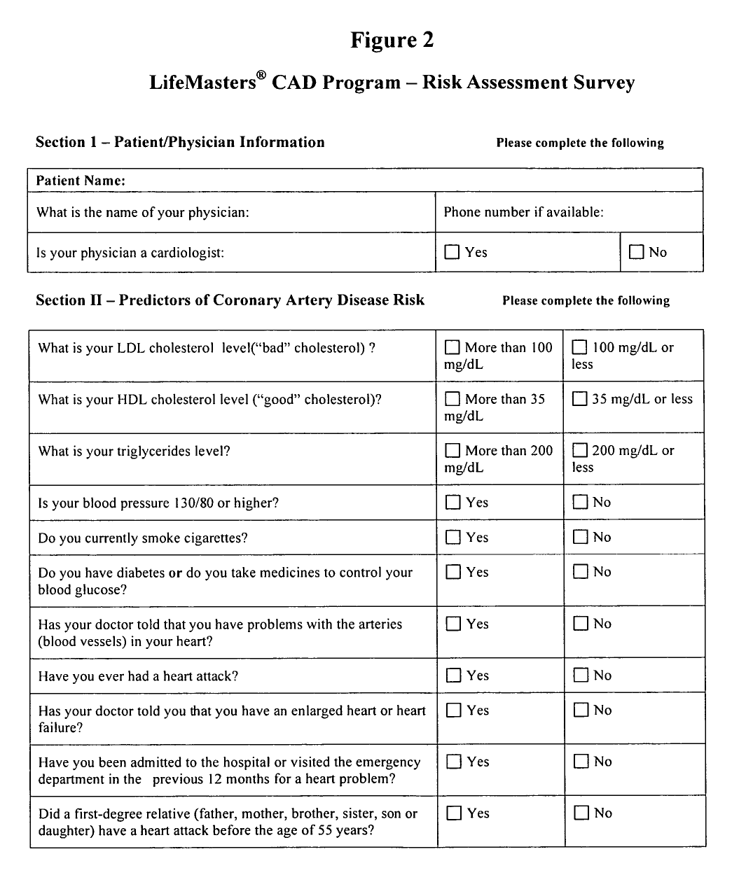

Once the population of patients has been defined, personal patient information is collected from the population of patients. The source of personal patient information is queried to gather information that is generally recognized by professional organizations as being associated with the preselected medical condition. In a preferred embodiment, the source of personal information is the patient themselves. By way of example, FIG. 2 is a model “risk assessment survey” for coronary artery disease, which may also be referred to herein as an “input collection tool”.

As shown, the type of information collected in the risk assessment survey can be adapted for use in assessing a patient's risk of having or developing a given medical condition. Collectively, categorizing the risk, for example as low-medium-high, as well as establishing that the patient most likely already has the medical condition, is referred to herein as “risk assessment”. For example, the survey depicted in FIG. 2 includes the following question:
“Has your doctor told you that you have problems with the arteries (blood vessels) in year heart?”

According to clinical guidelines, a “yes” answer to this question indicates that the patient already has coronary artery disease. Thus, this question is designed to identify patients that are “at risk” of having CAD or having a high likelihood of contracting CAD.

Patient Analysis Outcome

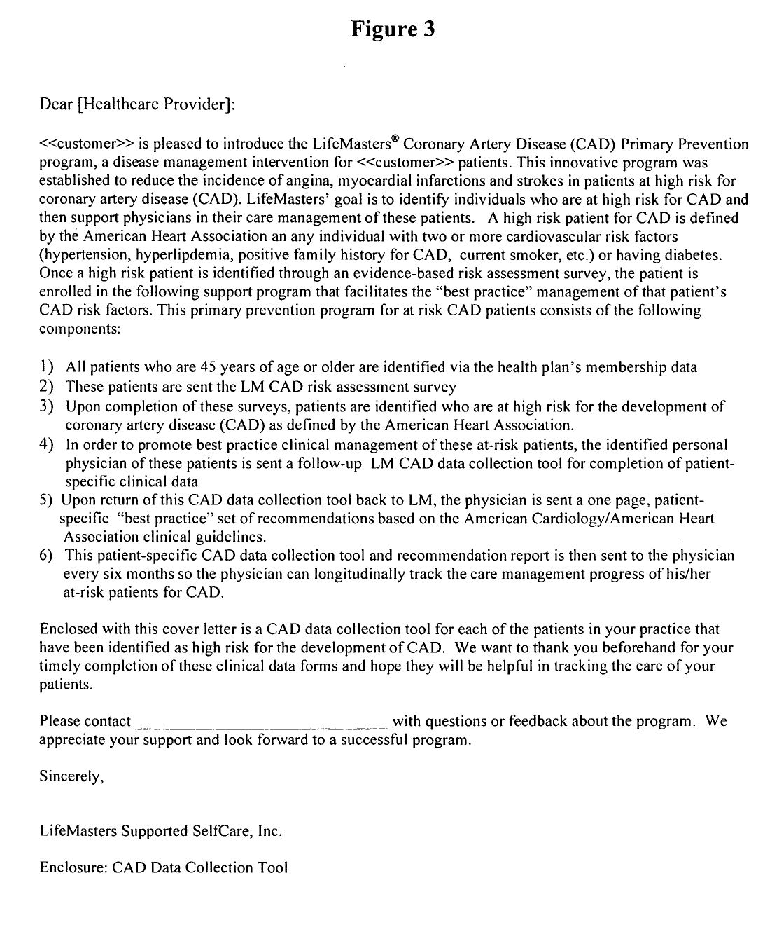

The next step in the system of the present invention is the input and processing of the personal patient information by the central system to generate the patient analysis outcome. The outcome can take any form, e.g., outcome that is visible on a computer screen or which may be printed and sent by mail or by fax to the healthcare provider. A sample letter that is sent to a physician following input and processing of personal patient information in a CAD system is shown in FIG. 3. As can be seen in FIG. 3, the outcome, which would be attached to the letter, may be as simple as the identification of individual patients within the patient population that are determined to have a “high risk” of contracting the medical condition based on the information that was supplied. Other types of outcome may include a more complete risk assessment of the entire population, such that individual members are categorized as having no risk, low risk, medium risk, high risk, or they are identified as already most likely having the medical condition.

Patient Management Information Input

This step of the system of the present invention is designed to collect information regarding the medical history of the patient that is relevant to the preselected medical condition. In particular, it is designed to establish how the patient's care has been managed in the past and/or is currently being managed with respect to the medical condition. This ensures that the recommendations outcome can be appropriately customized for individual patients and sufficiently complete to provide the healthcare provider with useful educational information for ongoing patient care.

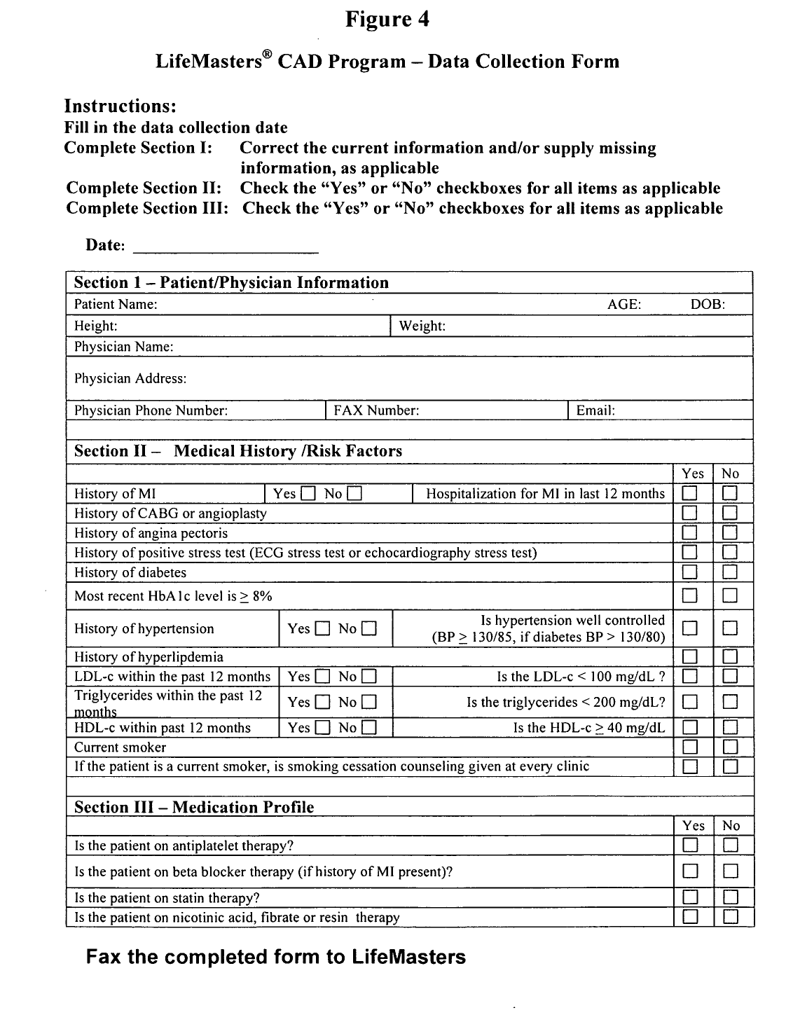

Accordingly, shown in FIG. 4, is an exemplary input collection device, a “Data Collection Form”, for use in a CAD system. Included therein are queries that are chosen according to clinical guidelines for customizing the recommendations outcome in a way that will be most useful to the healthcare provider in treating a CAD patient. For convenience, if the recipient of the patient analysis outcome and the entity from which the patient management information input is sought are the same, then the steps of providing this outcome and seeking input can take place simultaneously.

Recommendations Outcome

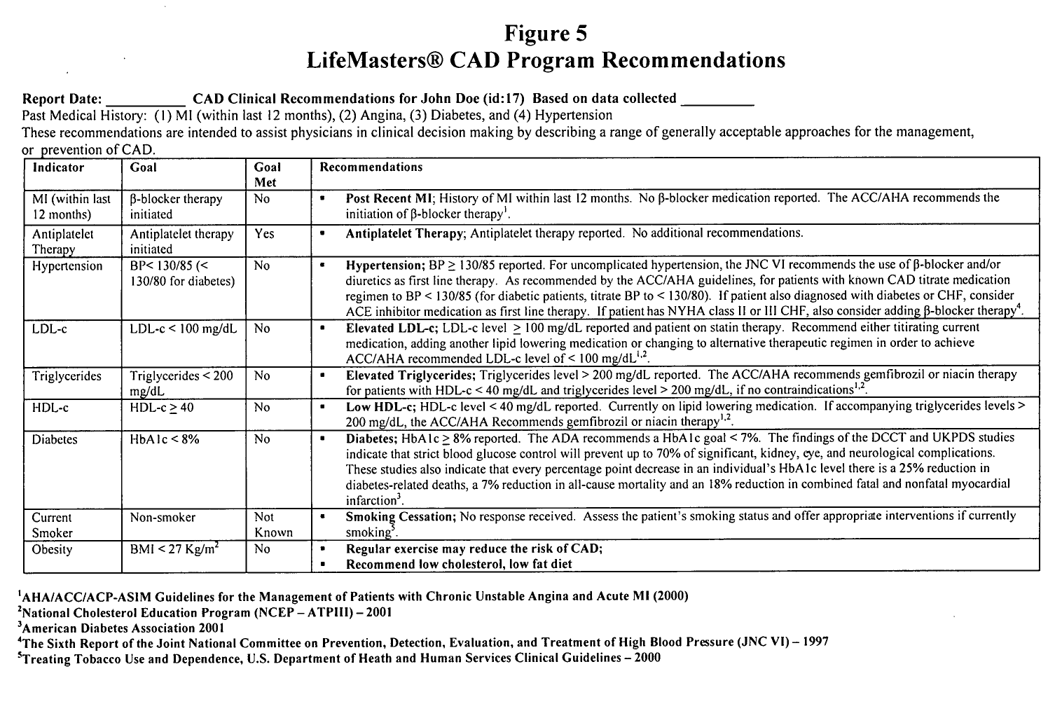

The next step in the system is the input and processing of the patient management information by the central system to generate the recommendations outcome. Shown in FIG. 5 is a sample recommendations outcome for a CAD patient. Unlike other methods designed to diagnose and prognose a patient's condition, the recommendations outcome provides a summary of patient-specific information and evidence-based clinical recommendations for the patient's ongoing management. Accordingly, the recommendations outcome is based on previous research-studies and clinical evidence that individuals whose input meets certain criteria would be most appropriately managed according to certain guidelines. Any “diagnostic” information about a patient is provided directly from the patient or healthcare provider when the patient-specific input is collected. Likewise, any prognostic analysis of the patient on the basis of the information collected is left completely to the healthcare provider.

In summary, the present invention is designed to rapidly and efficiently customize publicly available, evidence-based “best practice” recommendations for individual patients that would be impossible to do manually for any sizeable patient population. By providing healthcare professionals with such recommendations, they will be better educated and able to intervene when appropriate to improve patient care. In addition, once the individual members are “enrolled” in the system, the steps of obtaining patient medical information and providing recommendations outcome can be repeated at any given time interval (monthly, quarterly, semiannually, yearly, etc.) to provide healthcare professionals with continually updated best medical practice guidelines for ongoing patient management.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the field or any modes are intended to be within the scope of the following claims.

We claim:

1. A method for providing patient-specific best medical practices recommendations for a population of patients suspected of having individual members with a risk for at least one preselected medical condition using a central system adapted to customize the recommendations for the individual members, wherein said method comprises the steps of:
a) selecting a population of patients that is suspected of having a higher than normal risk for the preselected medical condition;
b) collecting personal patient information about the individual members that comprises factors that indicate risk of having or developing the preselected medical condition;
c) processing the personal patient information by the central system to provide a patient analysis outcome that reflects risk assessment of the preselected medical condition;
d) collecting patient management information about the individual members that comprises a medical history relevant to the preselected medical condition; and
e) processing the patient management information by the central system to provide a recommendations outcome that reflects best medical practices for future management of the individual members relative to the medical condition.

2. The method according to claim 1, wherein steps d) and e) are repeated at least once.

3. The method according to claim 1, wherein the preselected medical condition is coronary artery disease.

4. The method according to claim 1, wherein the preselected medical condition is diabetes.

5. The method according to claim 1, wherein the preselected medical condition is pulmonary disease.

6. The method according to claim 1, wherein the preselected medical condition is congestive heart failure.

7. The method according to claim 1, wherein the recommendations outcome is sent to a healthcare provider via mail, e-mail, or is accessible by a healthcare provider over an internet web page.

8. The method according to claim 1, wherein the central system is pre-programmed with algorithms that allow for customization of output based on professional associations' evidence-based best practices according to the individual member's patient management information.

9. The method of claim 1, wherein the personal patient information is collected using a risk assessment survey that is completed by the individual member.

10. The method of claim 1, wherein the patient management information is collected using data collection tool that is completed by a healthcare provider.

11. A system for providing patient-specific best medical practices recommendations for a population of patients suspected of having individual members with the risk for at least one preselected medical condition comprising a central system adapted to customize the recommendations for the individual members, or and said system comprises:
a) a CPU programmed to receive and analyze personal patient information that comprises factors that indicate risk of having or developing the preselected medical condition;
b) a first output device adapted to provide patient analysis outcome that reflects risk assessment of the preselected medical condition;
c) a CPU programmed to receive and analyze patient management information about the individual members that comprises a medical history relevant to the preselected medical condition; and
d) a second output device, wherein said second output device is the same or different than the first output device, adapted to provide recommendations outcome that reflects best medical practices for future management of the individual members relative to the medical condition.

12. The system according to claim 11, further comprising at least one central server that is accessible on line and at least one remote access terminal.

13. The system according to claim 11, wherein the output device of (b), (d) or both (b) and (d) further comprises a communications interface capable of transmitting output on-line.
